(19) United States
(12) Patent Application Publication
(10) Pub. No.: US 2025/0002883 A1
(43) Pub. Date: Jan. 2, 2025

(54) COMPOSITION FOR GENE EDITING AND USE THEREOF

(71) Applicants: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(72) Inventors: Taeyoung Koo, Seoul (KR); Dae Sik Kim, Suwon-si (KR); Eun Young Choi, Seoul (KR); Hye Yeon Hwang, Suwon-si (KR)

(21) Appl. No.: 18/758,713

(22) Filed: Jun. 28, 2024

(30) Foreign Application Priority Data
Jun. 29, 2023 (KR) 10-2023-0084418

(51) Int. Cl. C12N 9/22; C12N 9/78; C12N 15/11; C12N 15/90

(52) U.S. Cl. CPC C12N 9/22; C12N 9/78; C12N 15/11; C12N 15/907; C12Y 305/04002; C07K 2319/00; C12N 2310/20

(57) ABSTRACT

The present disclosure relates to a composition for gene editing and a use thereof. According to the present disclosure, an adenine base editor LbABE8e system in which a Cas12a protein variant including one or more mutations and adenine deaminase are fused has altered PAM specificity to achieve a significant genomic single base editing effect on target DNA, and thus expands the range of applicable CRISPR system selection. Accordingly, due to gene editing, it is expected that the adenine base editor LbABE8e system may be widely used in various fields, such as gene therapy, creation of commercial profits with industrial strains with improved productivity, improvement of the quality of public health care through improvement of intestinal microorganisms, and improvement of crops and livestock breeds free from GMO issues.

### CROSS-REFERENCE TO RELATED APPLICATIONS

This application is based on and claims priority from Korean Patent Application No. 10-2023-0084418, filed on Jun. 29, 2023, with the Korean Intellectual Property Office, the disclosure of which is incorporated herein in its entirety by reference.

### FIELD OF THE INVENTION

The present disclosure relates to a composition for gene editing and a use thereof.

### BACKGROUND OF THE INVENTION

Genetic scissoring technology using a CRISPR/Cas9 system has brought forth rapid development in current gene therapy technology.

Genetic scissors commonly induce DNA double strand breaks (DSBs). Living cells then recognize these DSBs as serious damage and activate a system to repair the damage. Specifically, since the speed of repair is fast, but does not depend on the original repair source, broken DNA strands are repaired through two pathways of non-homologous end-joining (NHEJ), which induces modification of various gene sequences, and homologous direct repair (HDR) that allows accurate recovery through template strands. The NHEJ pathway is usually used to remove genes, and the HDR pathway is usually used to correct or insert genes.

However, editing by HDR is known to be very inefficient, and in particular, DNA cleavage by Cas9 nuclease often causes insertion or deletion (indel) of unwanted DNA at a target site. To compensate for these shortcomings, recently, the CRISPR/Cas system has been used by removing the cleavage function and using only the feature that recognizes a specific DNA sequence. Various gene regulators are combined and used with a catalytically impaired Cas9 protein (dead Cas9; dCas9). In particular, by using base-editing genetic scissors (hereinafter also referred to as “a base editor”), which is called the 4th generation gene editing technology, it is possible to delete a gene or convert the gene into a desired trait by editing or replacing a specific sequence.

As the base editor, two types have been largely used. In other words, there are a cytidine base editor (CBE) capable of finding only cytosine (C) from a DNA sequence to be replaced with thymine (T) by binding cytidine deaminase to Cas9 nickase (nCas9) from which a double-stranded DNA cleavage function of CRISPR/Cas9 is removed (dCas9), or only a single-stranded DNA cleavage function is removed, and an adenine base editor (ABE) capable of replacing adenine (A) with guanine (G) by binding adenine deaminase. These base editors rarely produce DNA double-strand breaks (DSBs) and do not require a donor DNA template, and thus are widely used in a wide range of research fields, including plant genome engineering, mouse zygote engineering, and biomedical applications.

However, similarly to CRISPR nuclease, the base editors also exhibit off-target effects in whole single guide RNA (sgRNA)-dependent genome. In addition, the ABE was found to catalyze the conversion of cytosine in addition to adenine located in a target sequence motif. In addition, the CBE induces indiscriminate DNA deamination in the genome regardless of sgRNA, and both the CBE and ABE induce indiscriminate RNA deamination throughout the whole transcript in RNA transcript. To solve the problem, there have been attempts to use Cas9 effector engineering techniques or modify cytidine/adenine deaminase, but the problem is still not completely solved, and thus it is required to develop new base editors.

### SUMMARY OF THE INVENTION

In order to solve the problems, the present inventors introduced a Cas12a variant that induced specific mutations G532R and K595R into Cas12a nuclease and a Cas12a-based adenine base editor Cas12a-ABE, and as a result, found that the engineered LbCas12a and ABE8e specifically recognized a 5′-TTCN-3′ PAM sequence to exhibit an excellent gene editing effect, and then completed the present disclosure.

Accordingly, the present disclosure has been made in an effort to provide a Cas12a protein variant including one or more mutations.

In addition, the present disclosure has also been made in an effort to provide an adenine base editor in which a Cas12a protein variant including one or more mutations and adenine deaminase are fused.

In addition, the present disclosure has also been made in an effort to provide a composition for gene editing including an adenine base editor in which a Cas12a protein variant including one or more mutations and adenine deaminase are fused; and guide RNA.

In addition, the present disclosure has also been made in an effort to provide a gene editing method including contacting the composition for gene editing with a target sequence in vitro.

In addition, the present disclosure has also been made in an effort to provide a kit for gene editing including the composition for gene editing.

Other objects and advantages of the present disclosure will be more apparent by the following detailed description and claims.

The terms used in the present disclosure are used for the purpose of description only, and should not be construed to be limited. The singular expression includes the plural expression unless the context clearly dictates otherwise. In the present disclosure, it should be understood that term “comprising” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Further, unless otherwise contrarily defined, all terms used herein including technological or scientific terms have the same meanings as those generally understood by a person with ordinary skill in the art. Terms which are defined in a generally used dictionary should be interpreted to have the same meaning as the meaning in the context of the related art, and are not interpreted as ideal or excessively formal meanings unless otherwise defined in the present disclosure.

As used in the present invention, the terms “nucleic acid sequence”, “nucleotide sequence” and “polynucleotide sequence” include: oligonucleotide or polynucleotide, and fragments or portions thereof, and genomic or synthetic-origin DNA or RNA, which may be a single-strand or double-strand, and refers to a sense or antisense strand.

Hereinafter, the present disclosure will be described in detail.

According to an aspect of the present disclosure, there are provided a Cas12a protein variant including one or more mutations, an adenine base editor in which a Cas12a protein variant including one or more mutations and adenine deaminase are fused, and a composition for gene editing including an adenine base editor in which a Cas12a protein variant including one or more mutations and adenine deaminase are fused; and guide RNA.

As used in the present invention, the terms “edit”, “editing” or “edited” refer to a method of altering a nucleic acid sequence (e.g., a wild-type naturally occurring nucleic acid sequence or a mutated naturally occurring sequence) of polynucleotide by selectively deleting a specific genomic target or incorporating a new specific sequence using an externally supplied DNA template. Such a specific genomic target may include a chromosomal region, mitochondrial DNA, a gene, a promoter, an open reading frame, or any nucleic acid sequence, but is not limited thereto.

As used in the present invention, the term “genome editing”, unless otherwise specified, refers to editing, restoring, modifying, losing and/or altering a gene function by deletion, insertion, substitution, etc. of a nucleic acid molecule by Cas12a cleavage at a target site of the target DNA.

As used in the present invention, the term “base editors (BES)” refer to single base editing means, and more specifically, are constructed by fusing adenine deaminase to the N-terminus of a Cas12a protein variant including one or more mutations, and named adenine base editors (ABEs). While the BEs do not cause double-strand breaks, the ABEs edit adenine to guanine at specific sites.

As used in the present invention, the term “adenine base editors (ABEs)” were constructed by fusing random naturally occurring deaminase (ecTadA) to the N-terminus of the Cas12a variant to edit adenine to guanine. The types of ABEs include ABE8e, ABEmax, ABEmax-m, ABE8e-V106W, ABE8.17-m, etc. according to the version, but are not limited thereto, and the ABEs including “adenine deaminase and the Cas12a protein variant according to the present disclosure are used interchangeably with “LbABE8e variant”, “LbABE8e-G532R/K595R” or “LbABE8e-G532R/K595R variant”.

The “adenine deaminase” according to the present disclosure is an enzyme that is involved in the removal of the amino group from adenine and the production of hypoxanthine, and it is reported that the enzyme is rarely found in higher animals, but is present slightly in the muscles of cows, milk, and the blood of rats, and present mostly in the intestines of crayfish and insects.

The adenine deaminase may be adenine deaminase derived from any bacteria known in the art, as long as the purpose of the present disclosure may be achieved, preferably derived from a bacterial species selected from the group consisting of Escherichia coli, Staphylococcus aureus, Salmonella typhi, Shewanella putrefaciens, Haemophilus influenzae, Caulobacter crescentus and Bacillus subtilis, and most preferably derived from Escherichia coli (ecTadA), but is not limited thereto.

The Cas12a protein variant includes one or more mutated amino acid residues corresponding to positions selected from the group consisting of amino acid positions of a wild-type Cas12a protein of 11, 12, 13, 14, 15, 16, 17, 34, 36, 39, 40, 43, 46, 47, 50, 54, 57, 58, 111, 126, 127, 128, 129, 130, 131, 132, 133, 134, 135, 136, 157, 158, 159, 160, 161, 162, 163, 164, 165, 166, 167, 168, 169, 170, 171, 172, 173, 174, 175, 176, 177, 178, 532, 533, 534, 535, 536, 537, 538, 539, 540, 541, 542, 543, 544, 545, 546, 547, 548, 549, 550, 551, 552, 553, 554, 555, 556, 565, 566, 567, 568, 569, 570, 571, 572, 573, 574, 575, 592, 593, 594, 595, 596, 597, 598, 599, 600, 601, 602, 603, 604, 605, 606, 607, 608, 609, 610, 611, 612, 613, 614, 615, 616, 617, 618, 619, 620, 626, 627, 628, 629, 630, 631, 632, 633, 634, 635, 636, 637, 638, 642, 643, 644, 645, 646, 647, 648, 649, 651, 652, 653, 654, 655, 656, 676, 679, 680, 682, 683, 684, 685, 686, 687, 688, 689, 690, 691, 692, 693, 707, 711, 714, 715, 716, 717, 718, 719, 720, 721, 722, 739, 765, 768, 769, 773, 777, 778, 779, 780, 781, 782, 783, 784, 785, 786, 871, 872, 873, 874, 875, 876, 877, 878, 879, 880, 881, 882, 883, 884, and 1048, but is not limited thereto as long as the purpose of the present disclosure may be achieved. More preferably, the Cas12a protein variant includes one or more mutated amino acid residues corresponding to amino acid positions 532 and 595 of the wild-type Cas12a protein, and most preferably mutated amino acid residues corresponding to amino acid positions G532R and K595R of the wild-type Cas12a protein.

In addition, the Cas12a protein variant of the present disclosure is characterized by specifically recognizing PAM with a sequence TTCN (preferably, TTCC), compared to the wild-type Cas12a protein.

In addition, in the present disclosure, the Cas12a protein variant may be a variant of the Cas12a protein derived from any bacteria known in the art, as long as the purpose of the present disclosure may be achieved, preferably derived from a bacterial species selected from the group consisting of Lachnospiraceae bacterium, Francisella novicid, Francisella tularensis, Prevotella albensis, Butyrivibrio proteoclasticus, Peregrinibacteria bacterium, Parcubacteria bacterium, Smithella sp., Acidaminococcus sp., Candidatus Methanoplasma termitum, Eubacterium eligens, Moraxella bovoculi, Leptospira inadai, Porphyromonas crevioricanis, Porphyromonas macacae, Succinivibrio dextrinosolvens, Prevotella disiens, Flavobacterium branchiophilum, Helcococcus kunzii, Eubacterium sp., Flavobacterium sp., Prevotella brevis, Moraxella caprae, Bacteroidetes oral, Porphyromonas cansulci, Synergistes jonesii, Prevotella bryantii, Anaerovibrio sp., Butyrivibrio fibrisolvens, Candidatus Methanomethylophilus, Butyrivibrio sp., Oribacterium sp., Pseudobutyrivibrio ruminis, and Proteocatella sphenisci, and most preferably derived from Lachnospiraceae bacterium.

In addition, in the present disclosure, the adenine base editor may preferably be any one selected from the group consisting of ABE8e, ABEmax, ABEmax-m, ABE8e-V106W, and ABE8.17-m, and most preferably ABE8.

In addition, in the composition of the present disclosure, adenine (A) may be substituted with any one base selected from the group consisting of guanine (G), cytosine (C), and thymine (T).

In the present disclosure, motifs and editing window that specifically bind to the ABEs of the present disclosure, which are engineered to edit genes, are identified, and it is demonstrated that the genome of a target subject may be effectively and precisely edited in single base units.

The subject is not limited as long as the method of the present disclosure may be applied, but may be preferably a plasmid, a virus, a prokaryotic cell, an isolated eukaryotic cell, or a eukaryotic organism other than a human.

The eukaryotic cell may be a cell of yeast, mold, plant, insect, amphibian, mammal, etc. For example, the eukaryotic cell may be a cell cultured in vitro, a transplanted cell, a primary cultured cell, an in vivo cell, or a mammal cell including a human, which is generally used in the art, but is not limited thereto.

The transfection according to the present disclosure includes any method of introducing a target gene (nucleic acid) into an organism, a cell, a tissue or an organ, and may be performed by selecting a suitable standard technique according to a host cell, as known in the art.

Further, according to another aspect of the present disclosure, there is provided a gene editing method including contacting the above-described composition for gene editing of the present disclosure with a target sequence in vitro.

Further, according to yet another aspect of the present disclosure, there is provided a kit for gene editing including the above-described composition for gene editing.

Since the method and the kit of the present disclosure use the composition described above, the description of duplicated contents will be omitted in order to avoid excessive complexity of the present disclosure.

According to the exemplary embodiments of the present disclosure, the adenine base editor LbABE8e system in which the Cas12a protein variant including one or more mutations and adenine deaminase are fused has altered PAM specificity to achieve a significant genomic single base editing effect on target DNA, and thus expands the range of applicable CRISPR system selection. Accordingly, due to gene editing, it is expected that the adenine base editor LbABE8e system may be widely used in various fields, such as gene therapy, creation of commercial profits with industrial strains with improved productivity, improvement of the quality of public health care through improvement of intestinal microorganisms, and improvement of crops and livestock breeds free from GMO issues.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows the activity of wild-type LbCas12a at sites with non-canonical and canonical PAMs. Indel frequencies induced by LbCas12a at 60 endogenous target sites containing non-canonical and canonical PAMs in HEK293T cells are shown, and indel frequencies were measured by targeted deep sequencing. Black arrows indicate target sites containing TTTT PAM. Each dot represents the mean of biologically independent triplicate base sequences.

FIGS. 2A to 2D show the activity of engineered LbCas12a variants at sites with non-canonical and canonical PAMs having a T or V nucleotide in the fourth position. The activities of LbCas12a and variants thereof are shown at 62 target sites according to a nucleotide type (V or T) at the fourth position in (A) canonical and (B) non-canonical PAMs. (C) The activities of LbCas12a and variants thereof are shown at 32 target sites containing a T at the fourth position of non-canonical PAMs. (D) The heatmap shows the mean indel frequencies measured by targeted deep sequencing. In the graph, the mean indel frequencies±SEM are shown. p values were derived from a Student's two-tailed t test. * p<0.05; NS, not significant.

FIGS. 3A to 3G show adenine base editing effects using LbABE8e variants engineered at sites with non-canonical PAMs. The adenine base editing efficiencies induced by (A) wild-type LbABE8e and engineered LbABE8e including (B) G532R, (C) K595R, or (D) G532R/K595R mutations at 22 endogenous target sites was shown, and the LbCas12a-G532R/K595R generated indels at frequencies of 20% or higher, as measured by targeted deep sequencing in HEK293T cells. The mean base editing frequencies±SEM were shown in the graph. (E) A representative heatmap shows mean base editing frequencies measured by targeted deep sequencing. (F) The base editing window within a protospacer are shown at six genomic sites in HEK293T cells for wild-type LbABE8e and engineered LbABE8e including G532R, K595R, or G532R/K595R mutations. (G) The enhanced editing window of the LbABE8e variants at site 13 is shown. Each dot represents an individual data point. The mean editing frequencies±SEM are indicated.

FIG. 4 shows the results of comparing and analyzing the efficiency of LbABE8e-G532A and LbABE8e-K595A variants prepared as controls and an LbABE8e-G532R/K595R variant.

FIGS. 5A to 5E show that editing of triple oncogenic mutations by engineered LbCas12a inhibits abnormal cancer cell proliferation. (A) shows a schematic diagram of oncogenic mutations in HCT-15 colon cancer cells. Oncogenic mutations are shown in red and PAM sequences are shown in blue. (B) shows a schematic diagram showing vectors encoding LbCas12a-G532R/K595R and LbABE8e-G532R/K595R, and a CRISPR array with direct repeats of multiplexed crRNA-V1, -V2, and -V3. The multiplexed crRNA-V2 and -V3 include separators between spacer sequences as shown in the figure. In all the three versions, expression of a single transcript is controlled by the U6 promoter. (C) shows the activities of LbCas12a-G532R/K595R with multiplexed crRNA-V3 targeting APC, PIK3CA, and TP53 and (D) shows the activities of LbABE8e-G532R/K595R with multiplexed crRNA-V3 targeting APC, PIK3CA, and TP53 in HCT-15 cells. The mean base editing frequencies of the protospacer are shown. (E) shows the results of measuring cell viabilities using MTT assay after transfection of plasmids encoding LbCas12a-G532R/K595R or LbABE8e-G532R/K595R and a corresponding crRNA. “Mock” refers to cells treated with plasmids encoding LbCas12a-G532R/K595R or LbABE8e-G532R/K595R and a non-targeting multiplexed crRNA-V3. Error bars indicate SEM (n=3). p values were derived from a Student's two-tailed t test. * p<0.05, ** p<0.01, *** p<0.001; NS, not significant.

### DETAILED DESCRIPTION

Hereinafter, Examples are to describe the present disclosure in more detail, and it will be apparent to those skilled in the art that the scope of the present disclosure is not limited by these Examples in accordance with the gist of the present disclosure.

### Example 1. Preparation of LbABE8e Variants of the Present Disclosure

The present inventors prepared crRNA plasmids and LbABE8e variants as follows:

First, a target gene sequence was obtained from the Ensembl genome browser, and designed so that forward oligonucleotide and reverse oligonucleotide capable of binding to a 23 nucleotide target site may bind complementarily. Two oligonucleotides were annealed using a T100 temperature cycler, and a Bsal enzyme was added to a pRG2z empty vector into which a U6 promoter was inserted for cloning. The cleaved vector was purified using ExpinTMGelSV (GeneallBiotechnology, Korea) and ligated by adding annealed oligonucleotides and T4 DNA ligase while culturing at 25° C. The cloned vector was transfected into DH5α chemically competent E. coli, smeared on an LB agar medium containing 100 μg/ml of an ampicillin sodium salt, and cultured at 37° C. for 16 hours or more. The grown colonies were grown in the LB medium containing 100 μg/ml of the ampicillin sodium salt, and then the plasmid was extracted.

In addition, codon-optimized LbABE8e was prepared through a human codon-optimization method by referring to sequence information of the LbABE8e (Addgene #138504) plasmid, and then G532R, K595R, G532R/K595R variants were introduced to the LbABE8e plasmid via a site-directed metagenesis method. Subsequently, the variants were transfected into DH5α chemically competent E. coli and the plasmid was extracted. HEK293T cells were dispensed in a 24-well plate 24 hours before transfection, and transfected by adding 2 μg of the plasmid (1500 ng gRNA and 500 ng LbABE8e) and PEI to each well. After 48 hours, genomic DNA was extracted from the cells.

In addition, a library for next-generation sequencing was constructed as follows:

The on-target loci of genomic DNA were amplified through three steps of polymerase chain reaction (PCR) using the corresponding primers and high-fidelity DNA polymerase. The amplified PCR product was purified and constructed into a deep sequencing library. PCR primers were designed using Primer3 (https://primer3.ut.ee/) and IDT PrimerQuestTMTool (https://sg.idtdna.com/pages/tools/primerquest), and pooling libraries performed next generation sequencing (NGS) using MiniSeq (Illumina, San Diego, CA).

In addition, data analysis and statistical analysis were conducted as follows:

Base editing frequencies were analyzed using BEAnalyzer from CRISPR RGEN tools (http://www.rgenome.net/), which was a web-based CRISPR analysis tool based on JavaScript-based internal algorithm, respectively. The base editing efficiency occurring within genomic DNA was determined by (the base editing number/total number of reads) counted at the exact target site. All results were expressed as mean±SEM (standard error of the mean), and statistical analysis was performed using GraphPad Prism 9.11.

### Example 2. Confirmation of Non-Canonical PAM-Dependent Activity of Cas12a of the Present Disclosure

The present inventors examined the genome editing activity of LbCas12a at sites containing various non-canonical PAMs to determine whether the PAM sequence affected the activity of LbCas12a.

Briefly, a total of 60 crRNAs targeting the TP53, APC, and PIK3CA genomic loci were generated at sites containing non-canonical PAMs (TCTN, TTCN, TCCN, and CTCN) and canonical TTTN PAMs. Then, the plasmids encoding LbCas12a and the corresponding crRNA were transfected into HEK293T cells, and the genome editing efficiencies were measured by targeted deep sequencing.

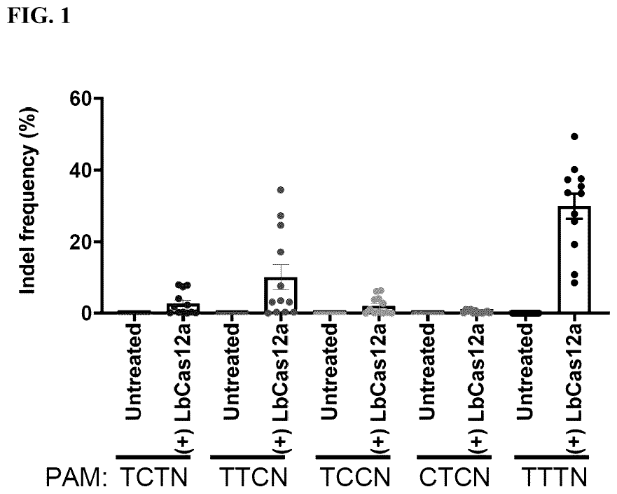

As a result, LbCas12a exhibited the activity at sites with TCTN, TTCN, TCCN, CTCN, and TTTN PAMs at frequencies of 2.7%±1.0%, 10.1%±3.6%, 2.1%±0.7%, 0.4%±0.1%, and 29.9%±3.5%, respectively. Interestingly, among the tested non-canonical PAMs, it was shown that LbCas12a recognized TTCN the best (FIG. 1).

### Example 3. Confirmation of PAM Preference by Cas12a Variants of the Present Disclosure

The present inventors examined whether the PAM sequences also affected the activities of LbCas12a variants including one or more mutations. At this time, the present inventors included two additional target sites with TTTT PAMs to identify statistically meaningful differences in PAM preferences.

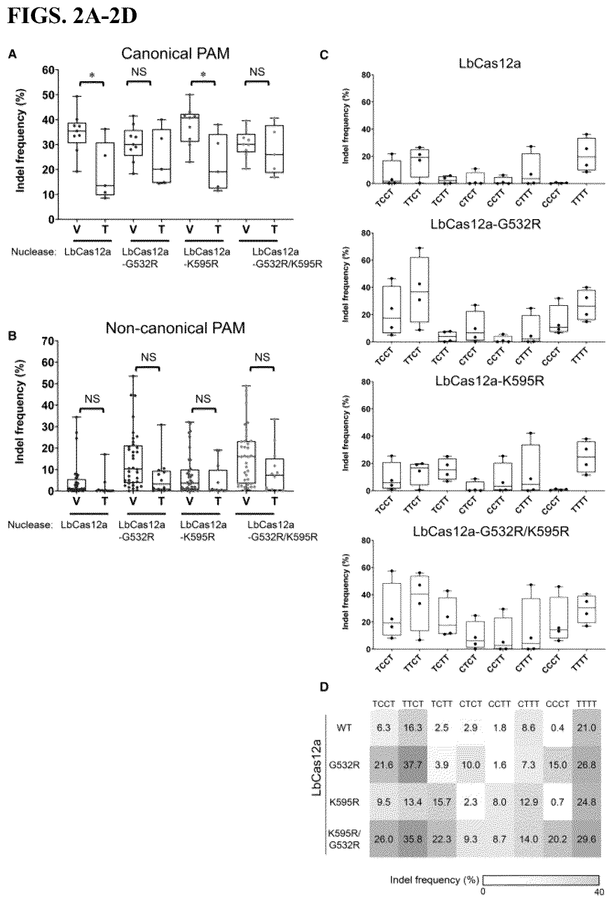

As a result, wild-type LbCas12a exhibited a preference for TTTV to TTTT PAMs, whereas neither LbCas12a-G532R nor LbCas12a-G532R/K595R exhibited a preference for the fourth nucleotide in canonical PAMs. In addition, the LbCas12a variants showed higher activity than wild-type LbCas12a at sites containing all tested non-canonical PAMs with a T in the fourth position (FIG. 2A).

In addition, interestingly, LbCas12a and its three variants showed no preference for the fourth nucleotide in the non-canonical PAMs (FIG. 2B).

Further, non-canonical PAMs (TCCT, TTCT, TCTT, CTCT, CCTT, CTTT, TTTT) recognized by wild-type LbCas12a induced mean indel frequencies in the range of 1.8% to 21%, whereas surprisingly, LbCas12a-G532R/K595R induced the mean indel frequency in the range of 8.7% to 35.8%, which showed that the activity of these variants was increased compared with that of wild-type LbCas12a (FIGS. 2C and 2D).

### Example 4. Confirmation of Gene Editing Effect by LbABE8e Variants of the Present Disclosure

Based on the results, in order to broaden the therapeutic utility by altering PAM specificity to expand the target range of base editors, the present inventors prepared LbABE8e variants in which one or more mutations were introduced into LbCas12a sites in LbABE8e which was an adenine base editor in which a Cas12a protein and adenine deaminase were fused, and examined whether to induce adenine base editing at sites containing various non-canonical PAMs, in order to confirm the gene editing effect by these variants. In other words, LbABE8e was a base editor consisting of a monomeric TadA-8e variant fused with catalytically dead LbCas12a in the LbCas12a protein.

In addition, the LbABE8e variants of the present disclosure were adenine base editors in which Cas12a protein variants including one or more mutations and adenine deaminase were fused.

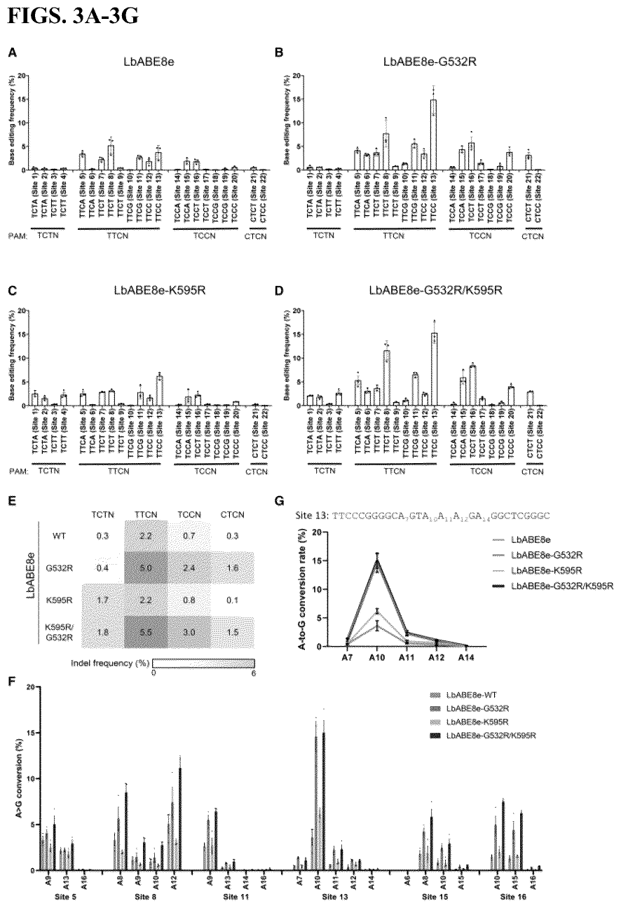

As a result, when the plasmids encoding wild-type LbABE8e and the corresponding crRNAs were transfected into HEK293T cells to determine the base editing efficiencies at 22 endogenous target sites where indels were generated by LbCas12a-G532R/K595R at frequencies 20% or higher, the base editing efficiency of wild-type LbABE8e varied from 0% to 5.3% at these sites with non-canonical PAMs (FIG. 3A).

Therefore, in order to overcome the low efficiency of wild-type LbABE8e in recognizing non-canonical PAMs, G532R and/or K595R mutations were introduced to human codon-optimized LbABE8e to prepare LbABE8e-G532R, LbABE8e-K595R, and LbABE8e-G532R/K595R, so as to alter PAM preference by suitable amino acid changes that loosen PAM constraints.

As a result, when introducing a single mutation (G532R) into wild-type LbABE8e, the base editing efficiency was increased at sites with several PAMs, while the effect of the K595R mutation in LbABE8e was minimal (FIGS. 3B and 3C). When LbABE8e-G532R/K595R was prepared by introducing mutations, the base editing efficiencies increased up to 14-fold compared with that of wild-type LbABE8e, at sites with all tested PAM sequences (FIGS. 3A and 3D). Among these, the effect of the G532R/K595R mutation was prominent in the site containing the TTCC PAM. At this site, the editing efficiency by wild-type LbABE8e was low, but LbABE8e-G532R/K595R showed high editing efficiency (FIGS. 3D and 3E).

In addition, the present inventors examined the base editing window of the LbABE8e variants at six genomic sites where A-to-G conversions were generated by LbABE8e-G532R/K595R at frequencies of 5% or more. The editing window for LbABE8e-G532R/K595R was similar to that of wild-type LbABE8e. The protospacer spans sites 8 to 14 (counted downstream of PAM) (FIG. 3F).

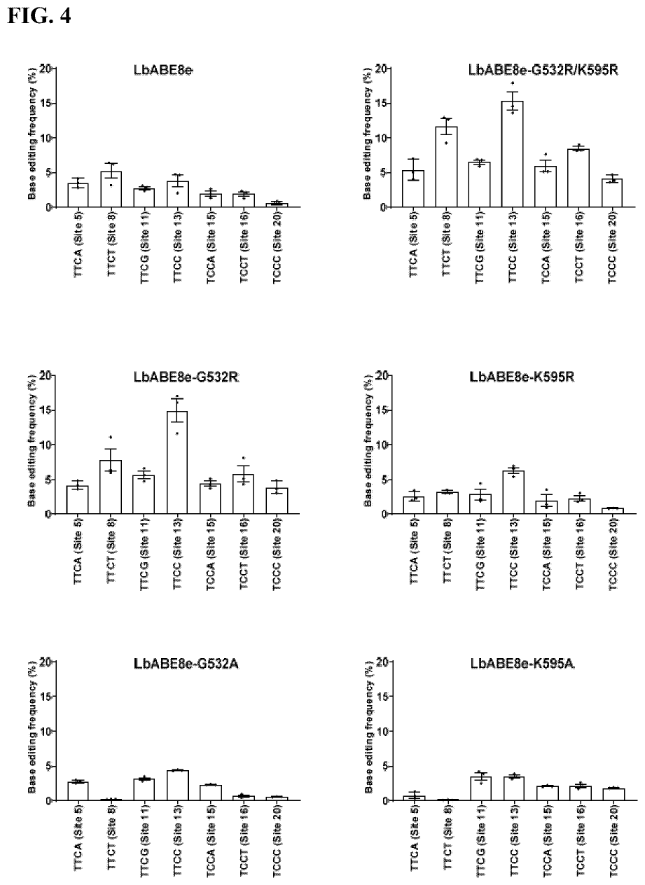

In addition, as controls, LbABE8e-G532A and LbABE8e-K595A variants were prepared and the efficiency was compared and analyzed with the LbABE8e-G532R/K595R variant. As a result, the gene editing improvement effect due to the introduction of the G532A or K595A variant into LbABE8e was not observed. On the other hand, the gene editing improvement effect was observed by introducing G532R/K595R into LbABE8e (FIG. 4).

That is, these results demonstrated that the LbABE8e-G532R/K595R variant of the present disclosure in which G532R/K595R was introduced into LbABE8e exhibited a significant gene editing improvement effect, as a result of comparing and analyzing the gene base editing efficiency of LbABE8e, LbABE8e-G532R, LbABE8e-K595R, LbABE8e-G532A, LbABE8e-K595A, and LbABE8e-G532R/K595R variants.

### Example 5. Confirmation of Oncogenic Gene Editing Effect by LbABE8e Variants of the Present Disclosure

The present inventors confirmed whether the gene editing induced by the LbABE8e-G532R/K595R variant, which induced the highest frequency of base editing among all LbABE8e variants was induced even in human cancer cell lines.

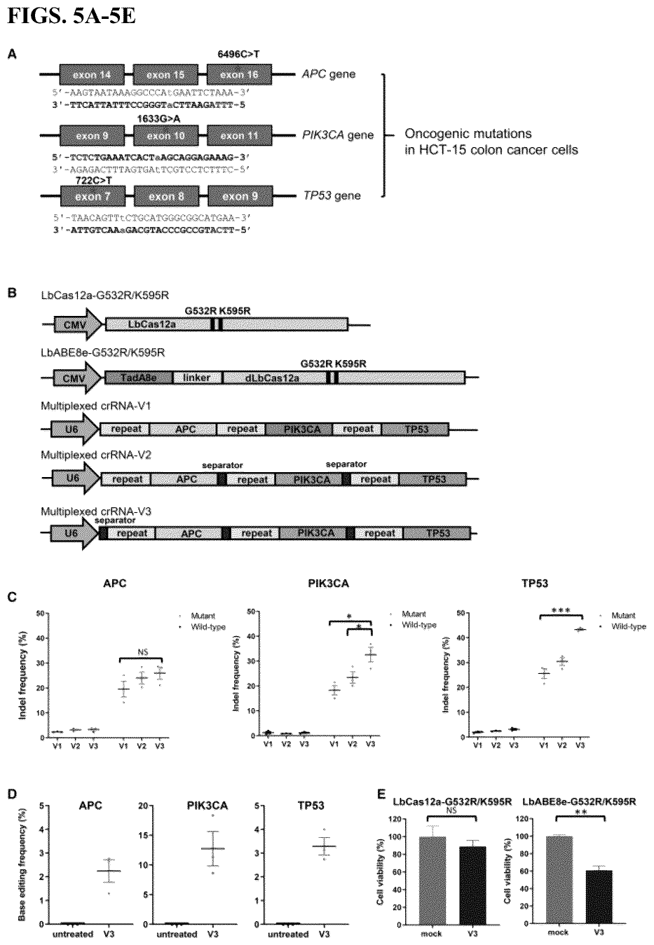

To examine oncogenic mutations present in HCT-15 Colorectal cancer (CRC) cells, the present inventors isolated genomic DNA from the cells and analyzed the sequences of cancer-associated genes using targeted deep sequencing. As a result, the present inventors found missense mutations in APC (6496C-to-T), PIK3CA (1633G-to-A), and TP53 (722C-to-T) that were present in 48%±0.2%, 41%±1.2%, and 50%±0.8% of the alleles, respectively (FIG. 5A). To evaluate LbCas12a-mediated editing of these triple oncogenic mutations, in each case, the present inventors designed several crRNAs that hybridized with these mutant target sequences located downstream of various non-canonical and canonical PAMs, and editing occurred at different frequencies depending on the PAM. When comparing genome editing efficiencies of the LbCas12a variants by transfecting each crRNA that resulted in the highest indel frequencies in the APC, PIK3CA, and TP53 genes into HCT-15 cells along with LbCas12a-G532R, LbCas12a-K595R, or LbCas12a-G532R/K595R, LbCas12a-G532R/K595R complexed with crRNAs induced mutant-allele-specific gene editing at the highest frequency.

Next, a CRISPR array targeting APC, PIK3CA, and TP53 simultaneously was generated by combining the respective crRNA-encoding sequences that resulted in the highest indel frequencies in each oncogene. The three crRNA-encoding sequences were arranged according to the GC content in the spacers, from lowest to highest: that targeting the APC (35% GC content), PIK3CA (39%), and TP53 (52%) genes. Multiplexed crRNA-V1 contained three spacer sequences and multiplexed crRNA-V2 contained separators between the spacers. Further, the present inventors further designed multiplexed crRNA-V3 including additional separators downstream of the promoter (FIG. 5B). After CRISPR array- and LbCas12a-G532R/K595R-encoding plasmids were transfected into HCT-15 cells, it was shown that multiplexed crRNA-V3 was associated with the highest activity, resulting in indel frequencies of 25.9%±2.4%, 32.6%±2.9%, and 43.3%±0.5%, and in mutant APC, PIK3CA, and TP53 alleles, the indel frequencies increased 1.7-fold and 1.3-fold compared with the activity associated with multiplexed crRNA-V1 and -V2, respectively (FIG. 5C).

In addition, when HCT-15 cells were treated with multiplexed crRNA-V3 and LbABE8e-G532R/K595R-encoding plasmids, adenine base editing was induced at frequencies of 2.3%±0.5% in APC, 12.8%±2.9% in PIK3CA, and 3.3%±0.4% in TP53, resulting in correction of oncogenic missense mutations (FIG. 5D). These results suggest that LbABE8e-G532R/K595R together with a multiplexed CRISPR array with three separators was used to successfully induce multiplexed gene editing.

### Example 6. Cancer Cell Proliferation Inhibitory Effect Mediated by Oncogenic Gene Editing by LbABE8e Variants of the Present Disclosure

In order to examine whether to reduce cancer cell proliferation by gene editing induced by the LbABE8e-G532R/K595R variant, which induced the highest frequency of base editing among all LbABE8e variants of the present disclosure, that is, LbCas12a-induced indels or LbABE8e induced base editing of these triple oncogenic mutant alleles could reduce cancer cell proliferation, the present inventors conducted an MTT cell proliferation assay.

As a result, HCT-15 cells treated with multiplexed crRNA-V3 and LbABE8e-G532R/K595R (LbABE8e variant) were significantly reduced in cell viability compared with a control (FIG. 5E). Taken together, these data showed that LbABE8e-mediated correction of multiple oncogenic mutations with a multiplexed CRISPR array efficiently inhibited abnormal cancer cell proliferation.

Overall, these results demonstrate that the adenine base editors (LbABE8e variant) of the present disclosure were constructed by fusing adenine deaminase (ecTadA) to the LbCas12a variant prepared by introducing the G532R/K595R mutation recognized with improved efficiency a specific non-canonical PAM sequence that was not recognized or recognized at a low efficiency by conventional wild-type LbABE8e to exhibit an excellent gene editing efficiency enhancing effect.

Therefore, the method of editing the genome at the single nucleotide level based on the LbABE8e system of the present disclosure provides important information that may become a basis to apply single nucleotide editing to various fields such as medicine and agriculture in the future.

As described above, specific parts of the present disclosure have been described in detail, and it will be apparent to those skilled in the art that these specific techniques are merely preferred exemplary embodiments, and the scope of the present disclosure is not limited thereto. Therefore, the substantial scope of the present disclosure will be defined by the appended claims and their equivalents.

What is claimed is:

1. A composition for gene editing comprising an adenine base editor in which a Cas12a protein variant including one or more mutations and adenine deaminase are fused; and guide RNA.

2. The composition for gene editing of claim 1, wherein the Cas12a protein variant includes one or more mutated amino acid residues corresponding to positions selected from the group consisting of amino acid positions of a wild-type Cas12a protein of 11, 12, 13, 14, 15, 16, 17, 34, 36, 39, 40, 43, 46, 47, 50, 54, 57, 58, 111, 126, 127, 128, 129, 130, 131, 132, 133, 134, 135, 136, 157, 158, 159, 160, 161, 162, 163, 164, 165, 166, 167, 168, 169, 170, 171, 172, 173, 174, 175, 176, 177, 178, 532, 533, 534, 535, 536, 537, 538, 539, 540, 541, 542, 543, 544, 545, 546, 547, 548, 549, 550, 551, 552, 553, 554, 555, 556, 565, 566, 567, 568, 569, 570, 571, 572, 573, 574, 575, 592, 593, 594, 595, 596, 597, 598, 599, 600, 601, 602, 603, 604, 605, 606, 607, 608, 609, 610, 611, 612, 613, 614, 615, 616, 617, 618, 619, 620, 626, 627, 628, 629, 630, 631, 632, 633, 634, 635, 636, 637, 638, 642, 643, 644, 645, 646, 647, 648, 649, 651, 652, 653, 654, 655, 656, 676, 679, 680, 682, 683, 684, 685, 686, 687, 688, 689, 690, 691, 692, 693, 707, 711, 714, 715, 716, 717, 718, 719, 720, 721, 722, 739, 765, 768, 769, 773, 777, 778, 779, 780, 781, 782, 783, 784, 785, 786, 871, 872, 873, 874, 875, 876, 877, 878, 879, 880, 881, 882, 883, 884, and 1048.

3. The composition for gene editing of claim 2, wherein the Cas12a protein variant includes one or more mutated amino acid residues corresponding to amino acid positions 532 and 595 of the wild-type Cas12a protein.

4. The composition for gene editing of claim 3, wherein the Cas12a protein variant includes mutated amino acid residues corresponding to amino acid positions G532R and K595R of the wild-type Cas12a protein.

5. The composition for gene editing of claim 1, wherein the Cas12a protein variant specifically recognizes PAM with a sequence TTCN compared to the wild-type Cas12a protein.

6. The composition for gene editing of claim 5, wherein the Cas12a protein variant specifically recognizes PAM with a sequence TTCC compared to the wild-type Cas12a protein.

7. The composition for gene editing of claim 1, wherein the Cas12a protein variant is derived from a bacterial species selected from the group consisting of Lachnospiraceae bacterium, Francisella novicid, Francisella tularensis, Prevotella albensis, Butyrivibrio proteoclasticus, Peregrinibacteria bacterium, Parcubacteria bacterium, Smithella sp., Acidaminococcus sp., Candidatus Methanoplasma termitum, Eubacterium eligens, Moraxella bovoculi, Leptospira inadai, Porphyromonas crevioricanis, Porphyromonas macacae, Succinivibrio dextrinosolvens, Prevotella disiens, Flavobacterium branchiophilum, Helcococcus kunzii, Eubacterium sp., Flavobacterium sp., Prevotella brevis, Moraxella caprae, Bacteroidetes oral, Porphyromonas cansulci, Synergistes jonesii, Prevotella bryantii, Anaerovibrio sp., Butyrivibrio fibrisolvens, Candidatus Methanomethylophilus, Butyrivibrio sp., Oribacterium sp., Pseudobutyrivibrio ruminis, and Proteocatella sphenisci.

8. The composition for gene editing of claim 7, wherein the Cas12a protein variant is derived from Lachnospiraceae bacterium.

9. The composition for gene editing of claim 1, wherein the adenine deaminase is derived from a bacterial species selected from the group consisting of Escherichia coli, Staphylococcus aureus, Salmonella typhi, Shewanella putrefaciens, Haemophilus influenzae, Caulobacter crescentus and Bacillus subtilis.

10. The composition for gene editing of claim 9, wherein the adenine deaminase is derived from Escherichia coli.

11. The composition for gene editing of claim 1, wherein the adenine base editor is any one selected from the group consisting of ABE8e, ABEmax, ABEmax-m, ABE8e-V106W, and ABE8.17-m.

12. The composition for gene editing of claim 11, wherein the adenine base editor is ABE8e.

13. The composition for gene editing of claim 1, wherein in the composition, adenine (A) is substituted with any one base selected from the group consisting of guanine (G), cytosine (C), and thymine (T).

14. A gene editing method comprising contacting the composition for gene editing of claim 1 with a target sequence in vitro.

15. A kit for gene editing comprising the composition for gene editing of claim 1.
